(19) United States
(12) Patent Application Publication
(10) Pub. No.: US 2018/0225427 A1
(43) Pub. Date: Aug. 9, 2018

(54) BRD2 PROTEIN INHIBITOR COMPOUNDS AND USES THEREOF

(71) Applicant: Insilico Medicine, Inc., Baltimore, MD (US)

(72) Inventors: Alexander M. Aliper, Ramenskoye (RU); Artem V. Artemov, Moscow (RU); Marine E. Bozdaganyan, Balashikha (RU)

(21) Appl. No.: 15/891,399

(22) Filed: Feb. 8, 2018

Related U.S. Application Data
(60) Provisional application No. 62/456,877, filed on Feb. 9, 2017.

(51) Int. Cl. G06F 19/00; C07D 207/34; C07D 233/90; C07D 401/06; A61P 35/00

(52) U.S. Cl. CPC G06F 19/706; C07D 207/34; C07D 233/90; C07D 401/06; G06F 19/707; G06F 19/701; G06F 19/704; G06F 19/705; A61P 35/00

(57) ABSTRACT

The present invention provides bioinformatics methods for predicting efficient protein inhibitors, compounds synthesized based on the methods and uses thereof, the method including identifying a BRD2 inhibitor by performing a pharmacophore-based search to produce a first plurality of candidate agents, applying a docking method to the first plurality of candidate agents to output a second plurality of candidate agents to provide at least one BRD2 inhibitor which are 2-acetyl-N,N,3-trisubstituted-5-[(substituted sulfonyl)substituted]imidazole-4-carboxamide and 1-{1,3,4-trisubstituted-5-[(substituted sulfonyl)substituted]-1H-pyrrol-2-yl}ethan-1-one, configured to inhibit the BRD2 protein.

### FIELD OF THE INVENTION

The present invention relates generally to bioinformatics methods for predicting efficient protein inhibitors, compounds synthesized based on the methods and uses thereof. More specifically, the present invention relates to bioinformatics methods for predicting BRD2 inhibitors, BRD2 inhibitor compounds and uses thereof.

### BACKGROUND OF THE INVENTION

Small molecules that influence the epigenetic status of cells have attracted attention as potential anticancer therapeutic agents, and many of these compounds are in preclinical and clinical trials (Dawson and Kouzarides 2012). Among the most promising targets of epigenetic drugs are bromodomain extra terminal (BET) proteins (Belkina and Denis 2012; Prinjha, Witherington, and Lee 2012).

The mammalian BET protein family includes ubiquitously expressed Brd2, Brd3, Brd4, and testis-specific BrdT. BET proteins contain two N-terminal bromodomains (BDs; BD1 and BD2) and the extra terminal (ET) protein interaction domain. Tandem BDs recognize acetylated lysines at histones, and this recognition interaction might recruit BET proteins to chromatin.

A distinct feature of BET proteins is that their attachment to chromatin persists during mitosis, which might help to reactivate postmitotic transcription and suggests a role in epigenetic memory (Garcia-Gutierrez, Mundi, and Garcia-Dominguez 2012). BDs can selectively distinguish specific acetylated lysines; for example, BD2 of Brd2 interacts preferentially with H4K12ac or H4K5ac/K12ac, whereas BD1 of the same protein recognizes H4K5ac/K8ac (Umehara et al. 2010; Filippakopoulos and Knapp 2012).

In addition, Brd2 BDs have much higher affinity to H4K5ac/K8ac/K12ac/K16ac tetraacetylated peptides than to single acetylation marks (Filippakopoulos et al. 2012; LeRoy et al. 2012). In contrast to diverse BD targets, ET domains, which are highly conserved among individual BET proteins (>80% identity among Brd2, Brd3, and Brd4), interact with the same proteins, for example, NSD3 methyltransferase or JMJD6 lysyl hydroxylase (Webby et al. 2009; Rahman et al. 2011).

BET proteins are found in multiple protein complexes; for example, Brd2 associates with RNA polymerase II and TATA box binding protein (TBP)-associated factors (TAFs), histone acetylases (CBP/p300), histone deacetylases, and chromatin assembly and remodeling factors (Denis et al. 2006). The BET proteins are transcription regulators, and the role of Brd3 and Brd4 in transcription is quite well established. Brd3 specifically binds the GATA1 transcription factor and enhances expression of GATA1-dependent genes (Gamsjaeger et al. 2011; Lamonica et al. 2011). Brd4 stimulates transcription via recruitment of active P-TEFb and induces phosphorylation of the C-terminal domain of RNA polymerase II (Hargreaves, Horng, and Medzhitov 2009; Devaiah et al. 2012).

However, less is known about the transcription regulation by Brd2. Brd2 associates with E2F transcription factors and helps to recruit TBP to promoters (Denis et al. 2006; Peng et al. 2006). In vitro, Brd2 enhances the passage of RNA polymerase II through acetylated chromatin within the gene body (LeRoy, Rickards, and Flint 2008). The importance of Brd2 is underscored by several experiments showing that Brd2 is an essential gene. Brd2−/− mice display defects in the development of the neural tube and abnormal brain structures and die during embryogenesis (Gyuris et al. 2009; Shang et al. 2009). Heterozygous Brd2+/− mice are viable but have a low number of inhibitory neurons in brain and develop spontaneous seizures (Velíšek et al. 2011). In addition, several studies link the BRD2 gene to epilepsy in humans (Pal et al. 2003; Cavalleri et al. 2007). Of interest, mice with lowered Brd2 expression are extremely obese but are protected from type 2 diabetes (Wang et al. 2009).

WO2015035112A1 describes cancer therapies, which combine epigenetic modulating agent(s) with immune modulating agent(s) These agents were identified to provide an improved treatment regimen over single agent therapy. In particular embodiments, the invention provides for improved treatment of NSCLC in patients via administration of exemplary immune modulating agents anti-PD-1 antibody or anti-PD-L1 antibody, which were observed to show enhanced activity in combination with the exemplary epigenetic modulating agent 5-deoxyazacytidine. Further, expression markers of responsive neoplastic cells are also disclosed.

There still remains a need for improved methods for identifying candidate agents for treating diseases.

There further remains a need for improved methods for identifying candidate agents and combinations of agents for treating proliferative diseases.

Additionally, there remains a need for improved agents and combinations of agents for treating proliferative diseases.

### SUMMARY OF THE INVENTION

It is an object of some aspects of the present invention to provide bioinformatics methods for predicting efficient protein inhibitors.

It is another object of some aspects of the present invention to provide bioinformatics methods for predicting efficient BRD2 protein inhibitors.

It is yet another object of some aspects of the present invention to provide efficient protein inhibitors.

It is yet a further object of some aspects of the present invention to provide efficient BRD2 protein inhibitors.

It is yet another object of some aspects of the present invention to synthesize efficient protein inhibitors.

It is yet a further object of some aspects of the present invention to synthesize efficient BRD2 protein inhibitors.

In some embodiments of the present invention, improved methods for identifying candidate agents for treating diseases are provided.

In some further embodiments of the present invention, improved methods for identifying candidate agents and combinations of agents for treating proliferative diseases are provided.

In some additional embodiments of the present invention, improved agents and combinations of agents for treating diseases are provided.

In some further additional embodiments of the present invention, improved agents and combinations of agents for treating proliferative diseases are provided.

In some embodiments of the present invention, improved bioinformatics methods are provided for predicting efficient protein inhibitors.

In some further embodiments of the present invention, improved bioinformatics methods are provided for predicting BRD2 protein inhibitors.

In other preferred embodiments of the present invention, a method and system is described for providing a BRD2 protein inhibitor.

In some embodiments of the present invention, a BRD2 protein inhibitor structure is predicted.

In some embodiments of the present invention, a BRD2 protein inhibitor structure is defined.

In additional embodiments of the present invention, a method of synthesis of a BRD2 protein inhibitor structure is provided.

In further embodiments of the present invention, methods of structure-based drug design are provided to find a new pharmaceutical compound that binds to a macromolecular receptor. Traditionally, design campaigns are carried out experimentally, a process that is both time-consuming and costly. Therefore, computational methods have been developed with the aim of reducing the time and cost to design new drugs. The choice of method depends on at which stage in the campaign it will be employed and there is typically a trade-off between accuracy and efficiency.

In the present invention, three main approaches for new drug searches are combined, namely:

a) a pharmacophore-based search; and

b) a docking method

The combination methods of the present invention are applied to rapid screening of relatively large collections of compounds.

The present invention provides bioinformatics methods for predicting efficient protein inhibitors, compounds synthesized based on the methods and uses thereof, the method including identifying a BRD2 inhibitor by performing a pharmacophore-based search to produce a first plurality of candidate agents, applying a docking method to the first plurality of candidate agents second plurality of candidate agents to provide at least three BRD2 inhibitors which are 2-acetyl-N-benzyl-4-(2-methanesulfonylphenyl)-1-methyl-1H-imidazole-5-carboxamide, 5-acetyl-N-benzyl-4-ethyl-2-(2-methanesulfonylphenyl)-1H-pyrrole-3-carboxamide, 1-[3-(2-hydroxyethyl)-5-(2-methanesulfonylphenyl)-4-[2-(pyridin-4-yl)ethynyl]-1H-pyrrol-2-yl]ethan-1-one configured to inhibit the BRD2 protein.

There is thus provided according to an embodiment of the present invention, a method for identifying a BRD2 inhibitor, the method including;

a. performing a pharmacophore-based search to produce a first plurality of candidate agents; and

b. applying a docking method to the first plurality of candidate agents to output a second plurality of candidate agents.

Notably, according to an embodiment of the present invention, the pharmacophore search was applied with at least one of the following rules;
a) No more than 5 hydrogen bond donors (the total number of nitrogen-hydrogen and oxygen-hydrogen bonds);
b) No more than 10 hydrogen bond acceptors (all nitrogen or oxygen atoms);
c) A molecular mass less than 500 daltons;
d) An octanol-water partition coefficient log P not greater than 5; and
e) A polar surface area no greater than 140 \acute{Å}².

Furthermore, according to an embodiment of the present invention, the pharmacophore search was applied with all of the following rules;
a) No more than 5 hydrogen bond donors (the total number of nitrogen-hydrogen and oxygen-hydrogen bonds);
b) No more than 10 hydrogen bond acceptors (all nitrogen or oxygen atoms);
c) A molecular mass less than 500 daltons;
d) An octanol-water partition coefficient log P not greater than 5; and
e) A polar surface area no greater than 140 \acute{Å}².

Additionally, according to an embodiment of the present invention, the at least one candidate agent is listed in Table 1.

Furthermore, according to an embodiment of the present invention, the at least one candidate agent is PDB 4UYG.

Moreover, according to an embodiment of the present invention, the at least one candidate agent is a compound of the formulae;

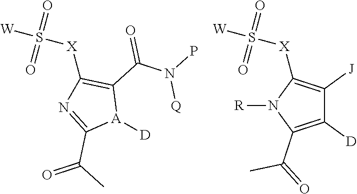

a derivative thereof, a salt thereof, a derivative salt thereof and combinations thereof.

Further, according to an embodiment of the present invention, the method identifies compounds including:
a)—X: Aromatic, Heteroaromatic ring;
b)—W: alkyl, heteroalkyl, cyclic alkyl;
c)—A: C or N or H;
d) D: H, alkyl, heteroalkyl;
e) P, Q: H, alkyl, cycloalkyl, aryl, alkyl or heteroaryl;
f) J: alkyl aryl, alkyl heteroaryl, unsaturated alkyl; and
g) R: H, alkyl.

Furthermore, according to an embodiment of the present invention, the compound has at least one of the following properties;
i. A molecular mass of less than 500;
ii. has less than 5 H-bond donors; and
iii. has less than 10 H-bond acceptors.

Importantly, according to an embodiment of the present invention, the compound has all of the following properties;
i. A molecular mass of less than 500;
ii. has less than 5 H-bond donors; and
iii. has less than 10 H-bond acceptors.

There is thus provided according to an embodiment of the present invention a compound according to any of following formulae:

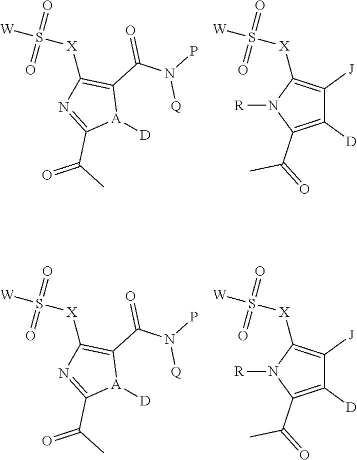

Furthermore, according to an embodiment of the present invention, the compound is a BRD2 inhibitor.

Moreover, according to an embodiment of the present invention, the compound includes
a)—X: Aromatic, Heteroaromatic ring;
b)—W: alkyl, heteroalkyl, cyclic alkyl;
c)—A: C or N or H;
d) D: H, alkyl, heteroalkyl;
e) P, Q: H, alkyl, cycloalkyl, aryl, alkyl or heteroaryl;
f) J: alkyl aryl, alkyl heteroaryl, unsaturated alkyl; and
g) R: H, alkyl.

Furthermore, according to an embodiment of the present invention, the compound has at least one of the following properties;
i. A molecular mass of less than 500;
ii. has less than 5 H-bond donors; and
iii. has less than 10 H-bond acceptors.

Importantly, according to an embodiment of the present invention the compound has all of the following properties;
i. A molecular mass of less than 500;
ii. has less than 5 H-bond donors; and
iii. has less than 10 H-bond acceptors.

Additionally, according to an embodiment of the present invention, the compounds described herein are suitable for administration to a subject to treat any disease or condition for which BET bromodomain inhibition provides clinical benefits.

Furthermore, according to an embodiment of the present invention, the disease or condition is selected from cancer, autoimmune disorder, respiratory disorder, asthma, cardiovascular disorder, neurological disease, Alzheimer's disease, inflammatory condition, sepsis and infection.

Moreover, according to an embodiment of the present invention, the compound is applied in combination with other therapeutic agents.

Further, according to an embodiment of the present invention, the compound, as described herein is used as immunotherapy agent to boost the patient's immune response against tumors or infection.

Yet further, according to an embodiment of the present invention the compound, as described herein, is administered to a patient in combination with immunotherapy agent(s).

Additionally, according to an embodiment of the present invention the immunotherapy agent(s) include one or many compounds or antibodies targeting PD-1, PD-L1 or CTLA4.

The present invention will be more fully understood from the following detailed description of the preferred embodiments thereof, taken together with the drawings.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified structural diagram of results of a pharmacophore search, in which spheres represent different pharmacophores, in accordance with an embodiment of the present invention;

FIG. 2 is a simplified structural diagram of a generalized chemical structural formula of compound, configured to inhibit a BRD2 protein, namely, 2-acetyl-N,N,3-tri substituted-5-[(substituted sulfonyl) substituted]imidazole-4-carboxamide and 1-{1,3,4-trisubstituted-5-[(substituted sulfonyl) substituted]-1H-pyrrol-2-yl}ethan-1-one, in accordance with embodiments of the present invention; and

FIGS. 3A-3C are simplified structural diagrams of chemical structural formula of compounds of the present invention, FIG. 3A shows 2-acetyl-N-benzyl-4-(2-methanesulfonylphenyl)-1-methyl-1 H-imidazole-5-carboxamide, FIG. 3B shows, 5-acetyl-N-benzyl-4-ethyl-2-(2-methanesulfonylphenyl)-1 H-pyrrole-3-carboxamide, and FIG. 3C shows 1-[3-(2-hydroxyethyl)-5-(2-methanesulfonylphenyl)-4-[2-(pyridin-4-yl)ethynyl]-1H-pyrrol-2-yl]ethan-1-one, wherein these three non-limiting examples of chemical compounds are configured to inhibit a BRD2 protein, in accordance with embodiments of the present invention.

In all the figures similar reference numerals identify similar parts.

### DETAILED DESCRIPTION OF THE MAIN EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

A structure-based design approach was used employing three-dimensional structural modeling of BRD2 protein active site and its binding relationship with inhibitor compounds to design the inventive compounds described herein. The crystal structures of the BRD2 protein were obtained from http://www.rcsb.org/pdb/home/home.do. The modeled protein molecules first were docked with their inhibitors with docking software.

The crystal structures of the BRD2 protein obtained from Protein Data Bank with natural ligand and inhibitors were used as templates for pharmacophore search via screening chemical databases. The active site of the BRD2 protein is well-known and conservative (Filippakopoulos et al., 2010), therefore all the structures for the search were chosen according to the its binding pocket and Lipinski rule of 5 (Al-Lazikani, 2004).

Docking into the site of binding allows calculating different energy contributions for ligand-receptor system. For example, on the basis of docking chemicals in the active site, it was determined that certain compound classes (4-[(2,2-disubstituted 2H-1,3-benzodioxol-5-yl)substituted]-6-ethane-1,2,5,7,8-pentasubstituted 1,2,3,4-tetrahydroquinoline) could replace acetylated lysine thereby allowing new hydrogen bonding and hydrophobic interactions within the pocket.

Docking procedure was provided with the usage of option of flexible chains (5 \acute{Å}) and the center for binding for each ligand was chosen according to the coordinates of active site of the protein. However, the possibility of allostery inhibition of the BRD2 protein was not taken into account.

### Example. Structure Analysis of 4UYG Protein-Ligand Complex

Crystal structure of BRD2 protein was taken from open access Protein Data Bank database (PDB code 4UYG). A pharmacophore search was performed according to the following rules:

1. No more than 5 hydrogen bond donors (the total number of nitrogen-hydrogen and oxygen-hydrogen bonds).

2. No more than 10 hydrogen bond acceptors (all nitrogen or oxygen atoms).

3. A molecular mass less than 500 daltons.

4. An octanol-water partition coefficient log P not greater than 5.

5. A polar surface area no greater than 140 \acute{Å}².

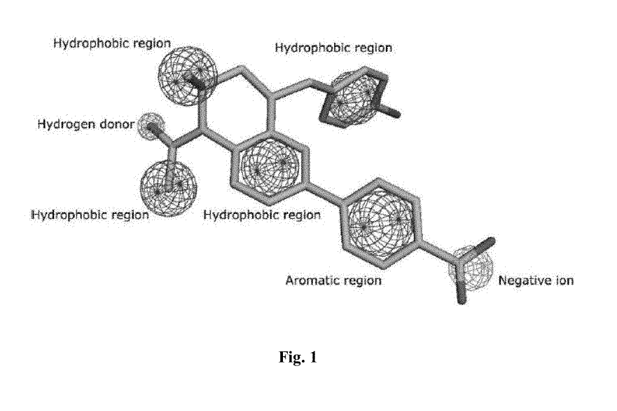

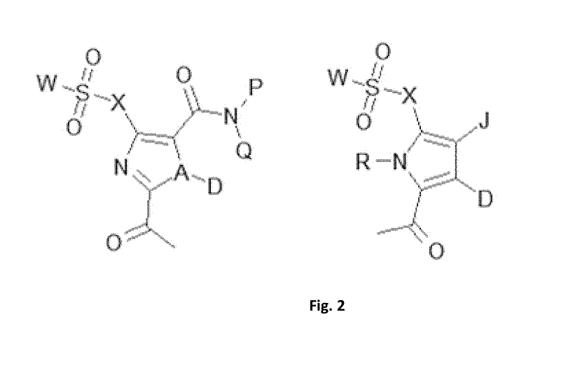

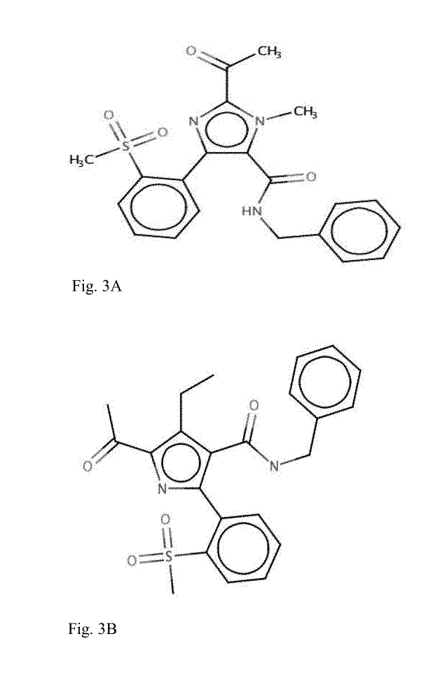

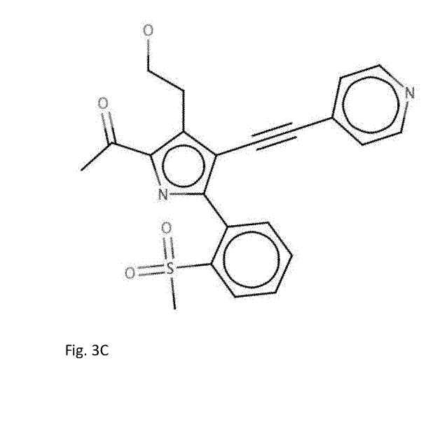

Pharmacophores were chosen as it's shown in FIG. 1. The search was made in PubChem Database as it has the largest number of molecules and conformations.

As a result, 16 hits were identified and 9 of them were chosen for the future analysis of the structures (Table 1). A docking procedure was provided as it was described before and molecules—potential inhibitors—as a template for the further design (highlighted in the table 2.1 with “+”). As the reference structure the one from the crystal structure was used and docked it into the binding site. The inhibitor has hydrogen bond with Asn429, Other interactions are mostly hydrophobic: with Met438, Pro371, Val435, Trp370, His433, Cys425 and Leu383. Also water molecule is coordinated inside the binding site forming hydrogen bonds with ligand and Tyr386.

As it can be seen from Table 1, the lowest energy has inhibitor from the crystal structure because protein structure is well optimized around it.

TABLE 1
Docking results.

| # | Compound PubChem ID | deltaG, kcal/mol |
|---|---|---|
| 1 | 77961323 | −7.82 |
| +2 | 1138973 | −8.85 |
| +3 | 52935565 | −9.7 |
| +4 | 78051210 | −8.8 |
| 5 | H00478661 | −7.7 |
| +6 | 223323 | −8.8 |
| 7 | 74830064 | −8.6 |
| 8 | AC1NCAKO | −7.1 |
| 9 | 01137466 | −8.3 |
| +10 | PDB 4UYG | −10.7 |

To identify new chemicals that satisfy these structural requirements, a de novo design approach was employed. Initially, a core of the molecule was chosen as structural analogs of PCID AC1NCAKO, PCID 01137466. Below, there is the list of non-limiting examples of possible derivatives of a candidate molecule.
a)—X: Aromatic, Heteroaromatic ring;
b)—W: alkyl, heteroalkyl, cyclic alkyl;
c)—A: C or N or H;
d)—D: H, alkyl, heteroalkyl;
e)—P, Q: H, alkyl, cycloalkyl, aryl, alkyl or heteroaryl;
f)—J: alkyl aryl, alkyl heteroaryl, unsaturated alkyl; and
g)—R: H, alkyl.

Then, all the molecules obtained were filtered with the following parameters:
1. Molecular mass less than 500.
2. Less than 5 H-bond donors.
3. Less than 10 H-bond acceptors.
4. Avoid duplicates and clashes.

As a result of the methods of the present invention, more than 119000 structures (combinations) were obtained.

Representative compounds of the present invention include:
2-acetyl-N-benzyl-4-(2-methanesulfonylphenyl)-1-methyl-1H-imidazole-5-carboxamide;
5-acetyl-N-benzyl-4-ethyl-2-(2-methanesulfonylphenyl)-1H-pyrrole-3-carboxamide;
1-[3-(2-hydroxyethyl)-5-(2-methanesulfonylphenyl)-4-[2-(pyridin-4-yl)ethynyl]-1H-pyrrol-2-yl]ethan-1-one;
2-acetyl-5-(4-methanesulfonylphenyl)-N,N,3-trimethyl-3H-pyrrole-4-carboxamide;
2-acetyl-4-[4-(ethanesulfonyl)phenyl]-1-ethyl-N,N-dimethyl-1H-imidazole-5-carboxamide;
2-acetyl-4-[4-(ethanesulfonyl)phenyl]-N,N,1-trimethyl-1H-imidazole-5-carboxamide;
2-acetyl-4-[4-(ethanesulfonyl)phenyl]-N,N-dimethyl-1H-imidazole-5-carboxamide;
2-acetyl-4-(4-cyclohexylmethanesulfonylphenyl)-1-ethyl-N-methyl-N-phenyl-1H-imidazole-5-carboxamide;
2-acetyl-4-[4-(cyclohexanesulfonyl)phenyl]-N,1-dimethyl-N-phenyl-1H-imidazole-5-carboxamide;
2-acetyl-4-[4-(cyclohexanesulfonyl)phenyl]-N-methyl-N-phenyl-1H-imidazole-5-carboxamide;
tris(2-acetyl-5-(4-cyclohexylmethanesulfonylphenyl)-3-ethyl-N-methyl-N-phenyl-3H-pyrrole-4-carboxamide);
2-acetyl-4-(4-cyclohexylmethanesulfonylphenyl)-1-ethyl-N-methyl-1H-imidazole-5-carboxamide;
2-acetyl-4-[4-(cyclohexanesulfonyl)phenyl]-N,1-dimethyl-1H-imidazole-5-carboxamide;
2-acetyl-4-[4-(cyclohexanesulfonyl)phenyl]-N-methyl-1H-imidazole-5-carboxamide;
tris(2-acetyl-5-(4-cyclohexylmethanesulfonylphenyl)-3-ethyl-N-methyl-3H-pyrrole-4-carboxamide);
2-acetyl-4-[4-(cyclopentanesulfonyl)phenyl]-1-methyl-1H-imidazole-5-carboxamide;
2-acetyl-4-[4-(cyclopentanesulfonyl)phenyl]-N, 1-dimethyl-1H-imidazole-5-carboxamide;
2-acetyl-4-[5-(cyclopentanesulfonyl)pyridin-2-yl]-1-methyl-1H-imidazole-5-carboxamide;
2-acetyl-5-[4-(cyclopentanesulfonyl)phenyl]-3-ethyl-3H-pyrrole-4-carboxamide;
2-acetyl-5-[5-(cyclopentanesulfonyl)pyridin-2-yl]-3-ethyl-3H-pyrrole-4-carboxamide;
2-acetyl-5-[5-(cyclopentanesulfonyl)pyridin-2-yl]-N-ethyl-3-methyl-3H-pyrrole-4-carboxamide;
1-{5-[4-(cyclohexanesulfonyl)phenyl]-1,3-dimethyl-4-(prop-2-en-1-yl)-1H-pyrrol-2-yl}ethan-1-one;
1-{5-[4-(cyclohexanesulfonyl)phenyl]-3,4-diethyl-1-methyl-1H-pyrrol-2-yl}ethan-1-one;
tetrakis(1-{5-[4-(cyclohexanesulfonyl)phenyl]-4-ethyl-1,3-dimethyl-1H-pyrrol-2-yl}ethan-1-one);
1-{5-[5-(cyclohexanesulfonyl)pyridin-2-yl]-3,4-diethyl-1-methyl-1H-pyrrol-2-yl}ethan-1-one;
bis(1-{5-[5-(cyclohexanesulfonyl)pyridin-2-yl]-4-ethenyl-3-methyl-1H-pyrrol-2-yl}ethan-1-one);
1-{5-[5-(cyclohexanesulfonyl)pyridin-2-yl]-4-ethyl-1,3-dimethyl-1H-pyrrol-2-yl}ethan-1-one;
bis(1-{5-[5-(cyclohexanesulfonyl)pyridin-2-yl]-4-ethyl-3-methyl-1H-pyrrol-2-yl}ethan-1-one);
1-[4-ethenyl-5-(5-methanesulfonylpyridin-2-yl)-3-methyl-1H-pyrrol-2-yl]ethan-1-one;
1-[4-ethyl-5-(4-methanesulfonylphenyl)-1,3-dimethyl-1H-pyrrol-2-yl]ethan-1-one;
1-[5-(4-methanesulfonylphenyl)-1,3,4-trimethyl-1H-pyrrol-2-yl]ethan-1-one;
1-{5-[4-(cyclopentanesulfonyl)phenyl]-3,4-diethyl-1-methyl-1H-pyrrol-2-yl}ethan-1-one;
bis(1-{5-[4-(cyclopentanesulfonyl)phenyl]-4-ethyl-1,3-dimethyl-1H-pyrrol-2-yl}ethan-1-one);
1-{5-[4-(ethanesulfonyl)phenyl]-1,3-dimethyl-4-(prop-2-en-1-yl)-1H-pyrrol-2-yl}ethan-1-one;
1-{5-[5-(cyclopentanesulfonyl)pyridin-2-yl]-3,4-diethyl-1-methyl-1H-pyrrol-2-yl}ethan-1-one;
1-{5-[5-(cyclopentanesulfonyl)pyridin-2-yl]-4-ethyl-3-methyl-1H-pyrrol-2-yl}ethan-1-one;
1-{5-[5-(ethanesulfonyl)pyridin-2-yl]-4-ethenyl-3-methyl-1H-pyrrol-2-yl}ethan-1-one;
1-{5-[5-(ethanesulfonyl)pyridin-2-yl]-4-ethyl-1,3-dimethyl-1H-pyrrol-2-yl}ethan-1-one;
1-{5-[5-(ethanesulfonyl)pyridin-2-yl]-4-ethyl-3-methyl-1H-pyrrol-2-yl}ethan-1-one
1-{5-[4-(cyclopropanesulfonyl)phenyl]-1,3,4-trimethyl-1H-pyrrol-2-yl}ethan-1-one;
1-{5-[4-(cyclopropanesulfonyl)phenyl]-1,3-dimethyl-4-(prop-2-en-1-yl)-1H-pyrrol-2-yl}ethan-1-one;
1-{5-[4-(cyclopropanesulfonyl)phenyl]-3,4-diethyl-1-methyl-1H-pyrrol-2-yl}ethan-1-one;
tris(1-{5-[4-(cyclopropanesulfonyl)phenyl]-4-ethyl-1,3-dimethyl-1H-pyrrol-2-yl}ethan-1-one);
1-{5-[5-(cyclopropanesulfonyl)pyridin-2-yl]-3,4-diethyl-1-methyl-1H-pyrrol-2-yl}ethan-1-one;
bis(1-{5-[5-(cyclopropanesulfonyl)pyridin-2-yl]-4-ethenyl-3-methyl-1H-pyrrol-2-yl}ethan-1-one);
1-{5-[5-(cyclopropanesulfonyl)pyridin-2-yl]-4-ethyl-1,3-dimethyl-1H-pyrrol-2-yl}ethan-1-one;
bis(1-{5-[5-(cyclopropanesulfonyl)pyridin-2-yl]-4-ethyl-3-methyl-1H-pyrrol-2-yl}ethan-1-one);
2-acetyl-4-[4-(cyclopropanesulfonyl)phenyl]-N,N,1-trimethyl-1H-imidazole-5-carboxamide;
2-acetyl-4-[4-(cyclopropanesulfonyl)phenyl]-N,N-dimethyl-1H-imidazole-5-carboxamide; and
2-acetyl-5-[4-(cyclopropanesulfonyl)phenyl]-N,N-dimethyl-3H-pyrrole-4-carboxamide.

### Embodiments

The following embodiments provide non-limiting examples of the present invention, which should not be deemed as limiting:

1. A method for identifying a BRD2 inhibitor, the method comprising:

a. performing a pharmacophore-based search to produce a first plurality of candidate agents; and

b. applying a docking method to said first plurality of candidate agents to output a second plurality of candidate agents.

2. A method according to embodiment 1, wherein said pharmacophore-based search comprises using a template selected from a crystal structures of a BRD2 protein, a natural ligand of a BRD2 protein and an inhibitor of a BRD2 protein.

3. A method according to embodiment 1, wherein said docking method comprises applying ligand-protein visual analysis software to said first plurality of candidate agents.

4. A method according to embodiment 1, wherein said docking method comprises applying docking software to said first plurality of candidate agents.

5. A method according to embodiment 2, wherein said pharmacophore search comprises at least one of the following rules:

a) No more than 5 hydrogen bond donors (the total number of nitrogen-hydrogen and oxygen-hydrogen bonds);

b) No more than 10 hydrogen bond acceptors (all nitrogen or oxygen atoms);

c) A molecular mass less than 500 daltons;

d) An octanol-water partition coefficient log P not greater than 5; and

e) A polar surface area no greater than 140 \acute{Å}².

6. A method according to embodiment 5, wherein said pharmacophore search comprises 2, 3, 4 or all of the following rules:

a) No more than 5 hydrogen bond donors (the total number of nitrogen-hydrogen and oxygen-hydrogen bonds);

b) No more than 10 hydrogen bond acceptors (all nitrogen or oxygen atoms);

c) A molecular mass less than 500 daltons;

d) An octanol-water partition coefficient log P not greater than 5; and

e) A polar surface area no greater than 140 \acute{Å}².

7. A method according to embodiment 5, wherein said at least one candidate agent is listed in Table 1.

8. A method according to embodiment 7, wherein said at least one candidate agent is PDB 4UYG.

9. A method according to embodiment 5, wherein said at least one candidate agent is a compound selected from one or more of the formulae:

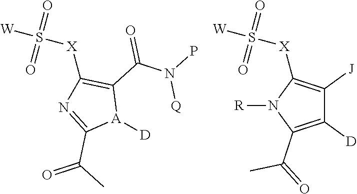

a derivative thereof, a salt thereof, a derivative salt thereof and combinations thereof.

10. A method according to embodiment 9, wherein

b)—X: Aromatic, Heteroaromatic ring;

c)—W: alkyl, heteroalkyl, cyclic alkyl;

d)—A: C or N or H

e)—D: H, alkyl, heteroalkyl

f)—P, Q: H, alkyl, cycloalkyl, aryl, alkyl or heteroaryl

g)—J: alkyl aryl, alkyl heteroaryl, unsaturated alkyl and

h)—R: H, alkyl.

11. A method according to embodiment 9, wherein said compound has at least one of the following properties:

i. A molecular mass of less than 500;

i. has less than 5 H-bond donors; and

ii. has less than 10 H-bond acceptors.

12. A method according to embodiment 9, wherein said compound has two or all of the following properties:

ii. A molecular mass of less than 500;

iii. has less than 5 H-bond donors; and

iv. has less than 10 H-bond acceptors.

13. A compound of any one or more of the following formulae:

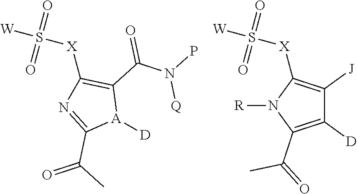

14. A compound according to embodiment 13, wherein said compound is a BRD2 inhibitor.

15. A compound according to embodiment 13, wherein
a)—X: Aromatic, Heteroaromatic ring;
b)—W: alkyl, heteroalkyl, cyclic alkyl;
c)—A: C or N or H
d)—D: H, alkyl, heteroalkyl
e)—P, Q: H, alkyl, cycloalkyl, aryl, alkyl or heteroaryl
f)—J: alkyl aryl, alkyl heteroaryl, unsaturated alkyl and
g)—R: H, alkyl.

16. A compound according to embodiment 13, wherein said compound has at least one of the following properties:

i. A molecular mass of less than 500;

ii. has less than 5 H-bond donors; and

iii. has less than 10 H-bond acceptors.

17. A compound according to embodiment 13, wherein said compound has two or all of the following properties:

i. A molecular mass of less than 500;

ii. has less than 5 H-bond donors; and

iii. has less than 10 H-bond acceptors.

18. A method wherein any compound of embodiments 13-17 is administered to treat any disease or condition for which BET bromodomain inhibition provides clinical benefits.

19. A method of embodiment 18, wherein a disease or condition is cancer, autoimmune disorder, respiratory disorder, asthma, cardiovascular disorder, neurological disease, Alzheimer's disease, inflammatory condition, sepsis or infection.

20. A method of embodiment 19, wherein a compound of embodiments 13-19 is applied in combination with other therapeutic agents.

21. A method of embodiments 19 or 20, wherein a compound of any of embodiments 13-17 is used as immunotherapy agent to boost the patient's immune response against tumors or infection.

22. A method of embodiments 18-21, wherein a compound of embodiments 13-17 is administered to a patient in combination with immunotherapy agent(s).

23. A method of embodiment 22, wherein immunotherapy agent(s) include one or many compounds or antibodies targeting PD-1, PD-L1 or CTLA4.

### REFERENCES

Al-Lazikani, Bissan. 2004. “Rule of Five (Lipinski Rule of Five).” In Dictionary of Bioinformatics and Computational Biology.
Belkina, Anna C., and Gerald V. Denis. 2012. “BET Domain Co-Regulators in Obesity, Inflammation and Cancer.” Nature Reviews. Cancer 12 (7): 465-77.
Cavalleri, Gianpiero L., Nicole M. Walley, Nicole Soranzo, John Mulley, Colin P. Doherty, Ashish Kapoor, Chantal Depondt, et al. 2007. “A Multicenter Study of BRD2 as a Risk Factor for Juvenile Myoclonic Epilepsy.” Epilepsia 48 (4): 706-12.
Dawson, Mark A., and Tony Kouzarides. 2012. “Cancer Epigenetics: From Mechanism to Therapy.” Cell 150 (1): 12-27.
Denis, Gerald V., Mark E. McComb, Douglas V. Faller, Anupama Sinha, Paul B. Romesser, and Catherine E. Costello. 2006. “Identification of Transcription Complexes That Contain the Double Bromodomain Protein Brd2 and Chromatin Remodeling Machines.” Journal of Proteome Research 5 (3): 502-11.
Devaiah, Ballachanda N., Brian A. Lewis, Natasha Cherman, Michael C. Hewitt, Brian K. Albrecht, Pamela G. Robey, Keiko Ozato, Robert J. Sims 3rd, and Dinah S. Singer. 2012. “BRD4 Is an Atypical Kinase That Phosphorylates serine2 of the RNA Polymerase II Carboxy-Terminal Domain.” Proceedings of the National Academy of Sciences of the United States of America 109 (18): 6927-32.
Filippakopoulos, Panagis, and Stefan Knapp. 2012. “The Bromodomain Interaction Module.” FEBS Letters 586 (17): 2692-2704.
Filippakopoulos, Panagis, Sarah Picaud, Maria Mangos, Tracy Keates, Jean-Philippe Lambert, Dalia Barsyte-Lovejoy, Ildiko Felletar, et al. 2012. “Histone Recognition and Large-Scale Structural Analysis of the Human Bromodomain Family” Cell 149 (1): 214-31.
Filippakopoulos, Panagis, Jun Qi, Sarah Picaud, Yao Shen, William B. Smith, Oleg Fedorov, Elizabeth M. Morse, et al. 2010. “Selective Inhibition of BET Bromodomains.” Nature 468 (7327): 1067-73.
Gamsjaeger, Roland, Sarah R. Webb, Janine M. Lamonica, Andrew Billin, Gerd A. Blobel, and Joel P. Mackay. 2011. “Structural Basis and Specificity of Acetylated Transcription Factor GATA1 Recognition by BET Family Bromodomain Protein Brd3.” Molecular and Cellular Biology 31 (13): 2632-40.
Garcia-Gutierrez, Pablo, Maria Mundi, and Mario Garcia-Dominguez. 2012. “Association of Bromodomain BET Proteins with Chromatin Requires Dimerization through the Conserved Motif B.” Journal of Cell Science 125 (Pt 15): 3671-80.
Gyuris, Aron, Diana J. Donovan, Kimberly A. Seymour, Lindsay A. Lovasco, Nathaniel R. Smilowitz, Anthony L. P. Halperin, Jan E. Klysik, and Richard N. Freiman. 2009. “The Chromatin-Targeting Protein Brd2 Is Required for Neural Tube Closure and Embryogenesis.” Biochimica et Biophysica Acta 1789 (5): 413-21.
Hargreaves, Diana C., Tiffany Horng, and Ruslan Medzhitov. 2009. “Control of Inducible Gene Expression by Signal-Dependent Transcriptional Elongation.” Cell 138 (1): 129-45.
Kumari, Rashmi, Rajendra Kumar, Open Source Drug Discovery Consortium, and Andrew Lynn. 2014. “G_mmpbsa—a GROMACS Tool for High-Throughput MM-PBSA Calculations.” Journal of Chemical Information and Modeling 54 (7): 1951-62.
Lamonica, Janine M., Wulan Deng, Stephan Kadauke, Amy E. Campbell, Roland Gamsjaeger, Hongxin Wang, Yong Cheng, et al. 2011. “Bromodomain Protein Brd3 Associates with Acetylated GATA1 to Promote Its Chromatin Occupancy at Erythroid Target Genes.” Proceedings of the National Academy of Sciences of the United States of America 108 (22): E159-68.
LeRoy, Gary, Iouri Chepelev, Peter A. DiMaggio, Mario A. Blanco, Barry M. Zee, Keji Zhao, and Benjamin A. Garcia. 2012. “Proteogenomic Characterization and Mapping of Nucleosomes Decoded by Brd and HP1 Proteins.” Genome Biology 13 (8): R68.
LeRoy, Gary, Brenden Rickards, and S. J. Flint. 2008. “The Double Bromodomain Proteins Brd2 and Brd3 Couple Histone Acetylation to Transcription.” Molecular Cell 30 (1): 51-60.
Pal, Deb K., Oleg V. Evgrafov, Paula Tabares, Fengli Zhang, Martina Durner, and David A. Greenberg. 2003. “BRD2 (RINGS) Is a Probable Major Susceptibility Gene for Common Juvenile Myoclonic Epilepsy.” American Journal of Human Genetics 73 (2): 261-70.
Peng, Jinhong, Wei Dong, Lu Chen, Tingting Zou, Yipeng Qi, and Yingle Liu. 2006. “Brd2 Is a TBP-Associated Protein and Recruits TBP into E2F-1 Transcriptional Complex in Response to Serum Stimulation.” Molecular and Cellular Biochemistry 294 (1-2): 45-54.
Pettersen, Eric F., Thomas D. Goddard, Conrad C. Huang, Gregory S. Couch, Daniel M. Greenblatt, Elaine C. Meng, and Thomas E. Ferrin. 2004. “UCSF Chimera?A Visualization System for Exploratory Research and Analysis.” Journal of Computational Chemistry 25 (13): 1605-12.
Prinjha, R. K., J. Witherington, and K. Lee. 2012. “Place Your BETs: The Therapeutic Potential of Bromodomains.” Trends in Pharmacological Sciences 33 (3): 146-53.
Rahman, Shaila, Mathew E. Sowa, Matthias Ottinger, Jennifer A. Smith, Yang Shi, J. Wade Harper, and Peter M. Howley. 2011. “The Brd4 Extraterminal Domain Confers Transcription Activation Independent of pTEFb by Recruiting Multiple Proteins, Including NSD3.” Molecular and Cellular Biology 31 (13): 2641-52.
Shang, Enyuan, Xiangyuan Wang, Duancheng Wen, David A. Greenberg, and Debra J. Wolgemuth. 2009. “Double Bromodomain-Containing Gene Brd2 Is Essential for Embryonic Development in Mouse.” Developmental Dynamics: An Official Publication of the American Association of Anatomists 238 (4): 908-17.
Srinivasan, Jayashree, Thomas E. Cheatham, Piotr Cieplak, Peter A. Kollman, and David A. Case. 1998. “Continuum Solvent Studies of the Stability of DNA, RNA, and Phosphoramidate—DNA Helices.” Journal of the American Chemical Society 120 (37): 9401-9.
Umehara, Takashi, Yoshihiro Nakamura, Moon Kyoo Jang, Kazumi Nakano, Akiko Tanaka, Keiko Ozato, Balasundaram Padmanabhan, and Shigeyuki Yokoyama. 2010. “Structural Basis for Acetylated Histone H4 Recognition by the Human BRD2 Bromodomain” The Journal of Biological Chemistry 285 (10): 7610-18.
Velíšek, Libor, Enyuan Shang, Jana Velíšgková, Tamar Chachua, Stephania Macchiarulo, Giorgi Maglakelidze, Debra J. Wolgemuth, and David A. Greenberg. 2011. “GABAergic Neuron Deficit as an Idiopathic Generalized Epilepsy Mechanism: The Role of BRD2 Haploinsufficiency in Juvenile Myoclonic Epilepsy.” PloS One 6 (8): e23656.
Wang, Fangnian, Hongsheng Liu, Wanda P. Blanton, Anna Belkina, Nathan K. Lebrasseur, and Gerald V. Denis. 2009. “Brd2 Disruption in Mice Causes Severe Obesity without Type 2 Diabetes.” Biochemical Journal 425 (1): 71-83.
Webby, Celia J., Alexander Wolf, Natalia Gromak, Mathias Dreger, Holger Kramer, Benedikt Kessler, Michael L. Nielsen, et al. 2009. “Jmjd6 Catalyses Lysyl-Hydroxylation of U2AF65, a Protein Associated with RNA Splicing.” Science 325 (5936): 90-93.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

1. A method for identifying a BRD2 inhibitor, the method comprising:
a. performing a pharmacophore-based search to produce a first plurality of candidate agents; and
b. applying a docking method to said first plurality of candidate agents to output a second plurality of candidate agents.

2. A method according to claim 1, wherein said pharmacophore-based search comprises using a template selected from a crystal structure of a BRD2 protein, a natural ligand of a BRD2 protein and an inhibitor of a BRD2 protein.

3. A method according to claim 1, wherein said docking method comprises applying ligand-protein visual analysis software to said first plurality of candidate agents.

4. A method according to claim 1, wherein said docking method comprises applying docking software to said first plurality of candidate agents.

5. A method according to claim 2, wherein said pharmacophore search comprises at least one of the following rules:
a) No more than 5 hydrogen bond donors (the total number of nitrogen-hydrogen and oxygen-hydrogen bonds);
b) No more than 10 hydrogen bond acceptors (all nitrogen or oxygen atoms);
c) A molecular mass less than 500 daltons;
d) An octanol-water partition coefficient log P not greater than 5; and
e) A polar surface area no greater than 140 \acute{Å}².

6. A method according to claim 5, wherein said pharmacophore search comprises all of the following rules:
a) No more than 5 hydrogen bond donors (the total number of nitrogen-hydrogen and oxygen-hydrogen bonds);
b) No more than 10 hydrogen bond acceptors (all nitrogen or oxygen atoms);
c) A molecular mass less than 500 daltons;
d) An octanol-water partition coefficient log P not greater than 5; and
e) A polar surface area no greater than 140 \acute{Å}².

7. A method according to claim 5, wherein said at least one candidate agent is listed in Table 1.

8. A method according to claim 7, wherein said at least one candidate agent is PDB 4UYG.

9. A method according to claim 5, wherein said at least one candidate agent is a compound of at least one of a formula: 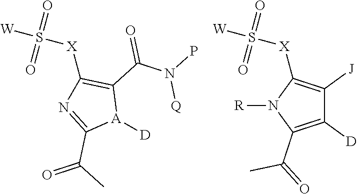
a derivative thereof, a salt thereof, a derivative salt thereof and combinations thereof.

10. A method according to claim 9, wherein
i)—X is selected from an aromatic or heteroaromatic ring;
a)—W is selected from alkyl, heteroalkyl and cyclic alkyl;
b)—A is selected from C, N and H
c)—D is selected from H, alkyl and heteroalkyl;
d)—P is selected from H, alkyl, cycloalkyl, aryl, alkyl and heteroaryl;
e), Q is selected from H, alkyl, cycloalkyl, aryl, alkyl and heteroaryl;
f)—J is selected from alkyl aryl, alkyl heteroaryl and unsaturated alkyl; and
g)—R is selected from H and alkyl.

11. A method according to claim 10, wherein said compound has at least one of the following properties:
i. a molecular mass of less than 500;
ii. has less than 5 H-bond donors; and
iii. has less than 10 H-bond acceptors.

12. A method according to claim 11, wherein said compound has all of the following properties:
i. a molecular mass of less than 500;
ii. has less than 5 H-bond donors; and
iii. has less than 10 H-bond acceptors.

13. A compound according to at least one of a following formula:

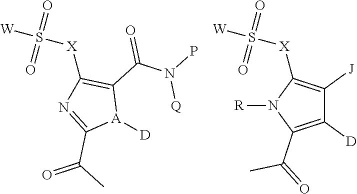

14. A compound according to claim 13, wherein said compound is a BRD2 inhibitor.

15. A compound according to claim 13, wherein
a)—X is selected from an aromatic or heteroaromatic ring;
b)—W is selected from alkyl, heteroalkyl and cyclic alkyl;
c)—A is selected from C, N and H;
d)—D is selected from H, alkyl and heteroalkyl;
e)—P is selected from alkyl, cycloalkyl, aryl, alkyl and heteroaryl;
Q is selected from H, alkyl, cycloalkyl, aryl, alkyl and heteroaryl;
g)—J is selected from alkyl aryl, alkyl heteroaryl and unsaturated alkyl; and
h)—R is selected from H and alkyl.

16. A compound according to claim 13, wherein said compound has at least one of the following properties:
i. a molecular mass of less than 500;
ii. has less than 5 H-bond donors; and
iii. has less than 10 H-bond acceptors.

17. A compound according to claim 13, wherein said compound has all of the following properties:
iv. A molecular mass of less than 500;
v. has less than 5 H-bond donors; and
vi. has less than 10 H-bond acceptors.

18. A method for treating a disease comprising administering a compound according to claim 13 in a pharmaceutically acceptable amount to a mammalian subject to treat a disease or condition for which BET bromodomain inhibition provides clinical benefits in said subject.

19. A method of claim 18, wherein said disease or said condition is selected from the group consisting of cancer, autoimmune disorder, respiratory disorder, asthma, cardiovascular disorder, neurological disease, Alzheimer's disease, inflammatory condition, sepsis and infection.

20. A method according to claim 18, wherein said compound is administered in combination with at least one other therapeutic agent.

21. A method according to claim 19, wherein said compound is an immunotherapy agent, effective in boosting an immune response against a tumor or infection in said mammalian subject.

22. A method according to claim 18, wherein said compound is administered to a said subject in combination with at least one immunotherapy agent.

23. A method according to claim 22, wherein said at least one immunotherapy agent is effective in targeting PD-1, PD-L1 or CTLA4.
